(19) United States
(12) Patent Application Publication
(10) Pub. No.: US 2020/0297377 A1
(43) Pub. Date: Sep. 24, 2020

(54) IMPROVED METHOD AND SYSTEM FOR NEEDLE GUIDE POSITIONING

(71) Applicant: INTIO, LLC, Boulder, CO (US)

(72) Inventor: MORGAN NIELDS, VAIL, CO (US)

(21) Appl. No.: 16/356,159

(22) Filed: Mar. 18, 2019

(51) Int. Cl. A61B 17/34; A61B 90/11; A61B 34/30; A61B 10/02; G06T 15/08

(52) U.S. Cl. CPC A61B 17/3403; A61B 90/11; A61B 34/30; A61B 2090/3762; G06T 15/08; A61B 2010/0208; A61B 10/0266

(57) ABSTRACT

Method and system embodiments are disclosed for improved positioning of one or more needle(s) relative to a patient tissue volume-of-interest (VOI) utilizing a robotically-positionable needle guide and enhanced imaging procedures. The embodiments include processing first and second sets of two-dimensional image data sets of the VOI, separately obtained by a computed tomography (CT) imaging device with a patient support platform maintained in a registered position relative to the CT imaging field and a corresponding predetermined frame of reference, with the robotically-positionable needle guide interconnected to the patient support platform, before and after placement of one or more needle(s) to obtain first and second sets of 3D image data. The first set of 3D image data is employed for determination of needle placement location(s). The first and second sets of 3D image data are registered (e.g. via deformable registration) and the registered 3D image data set is provided for generating an image display at a user interface to allow for user verification of desired needle placement.

### FIELD OF THE INVENTION

The present invention relates to improved positioning of one or more needle(s) employable for the removal of tissue (e.g. a biopsy sample) from a patient tissue volume-of-interest (VOI) or for the treatment of tissue within a patient tissue VOI (e.g. ablation of cancerous tissue).

### BACKGROUND

High precision needle placement is desirable in the medical field of tissue diagnosis and treatment of diseased tissue. In particular, suspicious tissue masses may be identified in patient tissue imaging procedures (e.g. “scanning” procedures) that indicate the presence of diseased tissue (e.g. cancerous tissue in the liver or other bodily regions). In turn, a tissue biopsy procedure may be completed in which one or more needle(s) is positioned relative to a suspicious tissue mass, wherein a tissue removal device is guided by the needle(s) for removal of one or more tissue sample(s) that is then analyzed for diagnosis of potential disease affecting the tissue. In order to achieve a reliable diagnosis, obtainment of a tissue sample(s) from one or more precise location(s) is desirable.

In instances of a suspicious tissue mass diagnosed as being diseased, treatment of such tissue mass may also entail the positioning of one or more needle(s) in the tissue mass, wherein a tissue treatment device is located by the needle(s) for administration of the desired treatment. In many instances, such treatment entails ablation of the suspicious tissue mass. In order to achieve a desired treatment outcome, e.g. ablative treatment of the entirety of a suspicious tissue mass with a desired peripheral margin, placement of one or needle(s) at a precise location(s) is desirable.

To date, imaging techniques have been developed that can provide high precision tissue image resolution, thereby allowing for improved identification of suspicious tissue masses. Further, tissue removal devices and tissue treatment devices have been developed that allow for improved removal and treatment of suspicious tissue masses.

Unfortunately, melding the improved tissue imaging and tissue removal/treatment developments has proven to be challenging, e.g. due to patient tissue movement and/or device movement that may occur between different ones of tissue imaging, needle placement, and tissue removal or tissue treatment procedures. In that regard, even small relative movements between a tissue mass of interest and imaging and/or needle placement devices can undermine realization of the desired accuracy. Such challenges are evidenced by situations in which a suspicious tissue mass is properly identified (e.g. via use of high resolution tissue images), but inaccurately biopsied for diagnosis (e.g. the sampled tissue is from a location outside of or on a margin of the suspicious tissue mass), and situations in which a diseased tissue mass is properly identified (e.g. via use of high resolution tissue images), but inaccurately treated (e.g. ablative treatment of less than all of the diseased tissue and/or with a desired peripheral margin).

### SUMMARY

The present disclosure is directed to method and system embodiments for improved positioning of at least one needle relative to a patient tissue volume-of-interest (VOI) that address the above-noted challenges of accurate needle placement. By way of example, the needle(s) may be of a type that is employed for guiding a device for the removal of tissue at a desired location (e.g. a tissue biopsy sample) and/or for guiding a device for the treatment of tissue at a desired location (e.g. tissue ablation). As may be appreciated, improved positioning of needle(s) in such applications can yield significant advantages in relation to accurate tissue diagnosis and desired tissue treatment.

Contemplated embodiments include a method for use in positioning a needle guide relative to a tissue volume-of-interest (VOI), and includes first processing (e.g. processing by a computer processor configured by executable computer code stored in non-transient memory)one or more first set(s) of two-dimensional (2D) image data sets obtained by a computed tomography (CT) imaging device with a patient positioned on and a robotically-positionable needle guide interconnected to a patient support platform that is positioned in a registered position relative to a CT imaging field of the CT imaging device and a corresponding predetermined frame of reference, to obtain one or more first set(s) of three-dimensional (3D) image data of the VOL In some arrangements, a plurality of first sets of 2D image data sets may be obtained by the CT imaging device for first processing to obtain a corresponding plurality of first sets of 3D image data in corresponding relation to a plurality of different imaging instances, e.g. CT imaging of the VOI without patient injection of a contrast bolus, CT imaging of the VOI with patient injection of a contrast bolus, CT imaging of the VOI at different power settings (e.g. different kV levels), and/or combinations thereof. The first processing may include first reconstructing the one or more first set(s) of 2D image data sets to obtain reconstructed one or more first set(s) of 2D image data sets as the one or more first set(s) of 3D image data (e.g. reconstructing by a computer processor configured by executable computer code comprising an image reconstruction algorithm stored in non-transient memory).

In such embodiments, a patient may be located on and the robotically-positionable needle guide may be interconnected to the patient support platform (e.g. supportably interconnected for movement with the patient support platform) at an initial location that is at least partially or entirely outside of the CT imaging field of the CT imaging device, wherein the support platform may then be subsequently positioned at the registered position relative to the CT imaging field of the CT imaging device and the predetermined frame of reference. In the later regard, the robotically-positionable needle guide may be advantageously operable for automated positioning relative to the VOI while the support platform is located in the registered position relative to the CT imaging field and predetermined frame of reference, free from manual positioning thereof. In some arrangements, the robotically-positionable needle guide may be provided so that portions thereof that may be locatable within the CT imaging field may be radiolucent, thereby facilitating in-field positioning of the robotically-positionable needle guide during imaging procedures. In some arrangements, the CT imaging device may be advantageously provided for cone beam CT.

The first set(s) of 3D image data may each include:
image data corresponding with a tissue mass-of-interest (TMOI) located in the VOI (e.g. a tissue mass identified as being potentially diseased);
image data corresponding with at least one anatomical structure located in the VOI and different than the TMOI; and
image data corresponding with a plurality of fiducial markers, wherein the robotically-positionable needle guide and the plurality of fiducial markers are disposed in predeterminable relation.
   In some arrangements, the plurality of fiducial markers may be supportably interconnected to the robotically-positionable needle guide in different corresponding locations (e.g. as part of an interconnected assembly and in radiolucent portions thereof).

The method may further include first generating first image display data utilizing the one or more first set(s) of 3D image data (e.g. generating by a computer processor configured by executable computer code stored in non-transient memory), wherein the first image display data may be provided for image display at a user interface in response to receipt of input indicative of at least one user-selected view of the VOI at the user interface. For each first set of 3D image data, the first generating may include the provision of first image display data in response to user input indicative of one or multiple user-selected views of the VOI at the user interface. The user selected view(s) may be two-dimensional and/or three-dimensional. The displayed view(s) may comprise a plurality of selected cross-sectional 2D views across the VOI and/or a plurality of selected perspective 3D views of the VOI, including dynamically generated 3D panning views. For such purposes, method may further include configuring the user interface to provide for user input and selection of one or multiple views of the VOI for display in relation to each first set of 3D image data (e.g. configuring by configuration of a computer processor by executable code). Where a plurality of first sets of 3D image data are generated/viewed, the method may further include receiving input indicative of a given user-selected set for further use as the first set of 3D image data. In turn, the method may further include configuring the user interface to provide for the user input and selection of a user-selected set for further use as the first set of 3D image data (e.g. configuring by configuration of a computer processor by executable code).

In some implementations, the first processing may further include first segmenting one or more of the reconstructed one or more first set(s) of 2D image data sets to obtain the one or more first set(s) of 3D image data (e.g. segmenting by a computer processor configured by executable computer code stored in non-transient memory). Such segmentation may be provided to provide enhanced image differentiation at different volume borders within the VOI at which tissue characteristics differ but may not be otherwise readily visible as such, and/or across tissue volumes within the VOI and within which tissue characteristics may be similar but not otherwise readily visible as such, thereby enhancing the ability to visually identify a TMOI and precise features thereof, including for example, volume border features and intra-volume vascular features (e.g. a periphery of a tissue volume corresponding with a cancerous tumor or other diseased tissue).

In some approaches, the first reconstructing and first segmenting of the first processing may be completed together. In other approaches, the first reconstructing may be completed, followed by the first segmenting.

In the later regard, the first reconstructing may be completed and the reconstructed one or more first set(s) of 2D image data sets may be provided for use as the one or more first set(s) of 3D image data in the first generating step, wherein corresponding first image display data may be provided for image display at the user interface. In turn, the first segmenting may include first applying a predetermined segmentation algorithm to one or more of the reconstructed one or more first set(s) 2D image data (e.g. applying by a computer processor configured by executable computer code comprising the predetermined segmentation algorithm stored in non-transient memory), in response to input of at least one user-selected location relative to at least one user-selected view of the VOI displayed at the user interface (e.g. selected by a user at the user interface). Such user-selected location, or “seed” location, may be a user-selected volume within the VOI that a user has identified in the displayed view as corresponding with the TMOI (e.g. a volume including at least a portion of the TMOI). In turn, the predetermined segmentation algorithm may provide for enhanced image differentiation of a tissue volume determined by the segmentation algorithm as having characteristics corresponding with those of the tissue located at the user-selected “seed” location. For example, the volume and/or volume borders of the determined tissue volume may be enhanced, or otherwise differentiated, in the segmented corresponding first set of 3D image data and provided for use in the first generating step, wherein upon display of one or multiple 2D and/or 3D views at the user interface, a user may visually assess the determined tissue volume that is enhanced in the displayed view(s), e.g. including dynamically generated 3D panning views, whereupon a user may select the segmented corresponding first set of 3D image data as the user-selected first set of 3D image date for further use.

In conjunction with such first segmenting, the method may further include receiving input indicative of a user-selected location, or volume, for segmentation. In turn, the method may further include configuring the user interface to provide for the user input and selection of the one or more user-selected location(s) for use in the application of the predetermined segmentation algorithm (e.g. configuring by configuration of a computer processor by executable code). As noted above, pursuant to the first segmenting, the segmented one or more first set(s) of 3D image data may be utilized in the first generating to provide corresponding first image display data to the user interface, wherein one of the segmented one or more first set(s) of 3D image data may be selected for use or otherwise used in the method. In that regard, the method may include configuring the user interface to provide for user selection of a segmented first set of 3D image data for further use (e.g. configuring by configuration of a computer processor by executable code).

The method may further include first determining needle guide positioning data utilizing the user-selected first set of 3D image data in response to input indicative of one or more user-selected needle placement location(s) relative to at least one or more corresponding user-selected view(s) across the VOI displayed at the user interface utilizing first image display data corresponding with the first set of 3D image data (e.g. determining by a computer processor configured by executable computer code stored in non-transient memory). In that regard, a user may select one or more needle placement location(s) that the user has determined desirable for guiding a tissue removal device or tissue treatment device to a desired location. The needle guide positioning data may include:
data indicative of user-selected needle insertion and inserted needle tip locations (e.g. 3D coordinates of each location relative to the predetermined frame of reference) corresponding with each of the one or more user-selected needle placement location(s) (i.e. for each a given needle, the 3D coordinates if the locations at which a user selects for needle entry in to patient tissue and an “end-point” needle tip location within the patient tissue); and,
data indicative of locations of the plurality of fiducial markers (e.g. 3D coordinates of each location relative to the predetermined frame of reference) in corresponding relation to each of the one or more user-selected needle placement location(s).
   In conjunction with the first determining, the method may further include receiving input from the user interface indicative of the one or more user-selected needle placement location(s). In turn, the method may further include configuring the user interface to provide for the user input and selection of the one or more user-selected needle placement location(s) (e.g. configuring by configuration of a computer processor by executable code).

The needle guide positioning data may be provided for automated positioning of the robotically-positionable needle guide relative to the VOI, free from manual positioning thereof. As such, the automated positioning may be advantageously completed with the patient support platform (i.e. with the patient positioned thereupon and the robotically-positionable needle guide interconnected thereto)located at the registered position relative to the CT imaging field of the CT imaging device and the predetermined frame of reference (e.g. maintained in the registered position after initial positioning), thereby facilitating the realization of enhanced positioning of one or more needle(s) in relation to the user-selected needle placement location(s).

In contemplated embodiments, the one or more needle(s) may then be located in the VOI in corresponding relation to the one or more user-selected needle placement location(s) comprising the needle guide positioning data. Such needle placement may be automated, manual, and/or a combination thereof. For example, the automated positioning of the robotically-positionable needle guide may be provided to locate the robotically-positionable needle guide so that the one or more needle(s) may be guided thereby to the corresponding one or more user-selected needle placement location(s) (e.g. corresponding with the 3D coordinates of the user-selected needle insertion and inserted needle tip locations). In some applications (e.g. tissue biopsy and tissue treatment applications), the automated positioning of the robotically-positionable needle guide may be provided to successively locate the robotically-positionable needle guide to a plurality of different locations so that each of a plurality of needles may be successively guided thereby to a corresponding plurality of different user-selected needle placement locations.

The method may further include, e.g. after positioning of the one or more needle(s) at the corresponding one or more user-selected needle placement location(s), second processing at least one second set of 2D image data sets obtained by the computed tomography (CT) imaging device (e.g. processing by a computer processor configured by executable computer code stored in non-transient memory), with the patient support platform (i.e. with the patient positioned thereupon and the robotically-positionable needle guide interconnected thereto) located at the registered position relative to the imaging field of the CT imaging device and the predetermined field of reference (e.g. maintained in the registered position after initial positioning), to obtain a second set of 3D image data of the VOL The second processing may include second reconstructing the at least one second set of 2D image data sets to obtain the second set of 3D image data (e.g. reconstructing by a computer processor configured by executable computer code stored in non-transient memory). The second set of 3D image data may include:
image data corresponding with the tissue mass-of-interest (TMOI) located in the VOI;
image data corresponding with the at least one anatomical structure located in the VOI and different than the TMOI;
image data corresponding with the plurality of fiducial markers; and,
image data corresponding with the one or more needle(s) located in to the VOI utilizing the robotically-positionable needle guide after the automated positioning of the robotically-positionable needle guide relative to the VOI.

The method may further include first registering the first and second sets of 3D image data of the VOI to obtain a first registered 3D image data set of the VOI (e.g. registering by a computer processor configured by executable computer code stored in non-transient memory). In some arrangements, the first registering may include deformable 3D registration processing of the first and second sets of 3D image data of the VOI (e.g. processing by a computer processor configured by executable computer code stored in non-transient memory), utilizing at least the corresponding image data of the first and second sets of 3D image data corresponding with the at least one anatomical structure, and optionally the image data corresponding with the plurality of fiducial markers, to obtain the first registered 3D image data set of the VOL Such deformable registration may further account for relative positional changes of common features of the first and second sets of 3D image data of the VOI.

After the first registering, the method may include second generating second image display data utilizing the first registered 3D image data set (e.g. generating by a computer processor configured by executable computer code stored in non-transient memory), wherein the second image display data is provided for display in response to input indicative of at least one user-selected view of the VOI at the user interface. The second generating may include the provision of second image display data in response to input indicative of multiple user-selected views of the VOI at a user interface. The user selected view(s) may be two-dimensional and/or three-dimensional. The displayed view(s) may comprise a plurality of selected cross-sectional 2D views across the VOI and/or a plurality of selected perspective 3D views of the VOI, including dynamically generated 3D panning views. For such purposes, the method may further include configuring the user interface to provide for user input and selection of one or multiple views of the VOI for display in relation to the first registered 3D image data (e.g. configuring by configuration of a computer processor by executable code).

The second image display data may be displayed so that a user may visually determine the desirability of the location of the one or more needle(s) in the VOL In turn, if the user is dissatisfied with the location of the one or more needle(s), the step of first determining needle guide positioning data may be repeated in response to the receipt of input indicative from the user interface indicative of one or more revised user-selected needle placement location(s) relative to at least one user-selected view across the VOI displayed at the user interface to obtain the needle guide positioning data. In turn, the second obtaining, second processing, first registering, and second generating steps may be repeated.

As noted, automated positioning of the robotically-positionable needle guide may be advantageously completed while the support platform (i.e. with the patient positioned thereupon and the robotically-positionable needle guide interconnected thereto) located at the registered position relative to the imaging field of the CT imaging device and the predetermined field of reference (e.g. maintained in the registered position after initial positioning). In turn, the first processing, first generating, first determining, second processing, first registering and second generating steps may be completed with the patient support platform positioned at the registered position relative to the imaging field of the CT imaging device and the predetermined field of reference a single time.

In contemplated embodiments, after the second generating, one or more tissue removal or tissue treatment device(s) may be located by the one or more needle(s) located in the VOI for tissue removal from or tissue treatment of the TMOI. For example, the tissue removal or tissue treatment device(s) may be advanced in to an exposed, open end of the one or more needle(s) and guided thereby for tissue removal from or tissue treatment of the TMOI in an automated, manual, and/or semi-automated manner.

In contemplated embodiments, after the tissue removal from or tissue treatment of the TMOI, the method may further include third processing at least one third set of 2D image data sets obtained by the computed tomography (CT) imaging device (e.g. processing by a computer processor configured by executable computer code stored in non-transient memory), with the patient support platform (i.e. with the patient positioned thereupon and the robotically-positionable needle guide interconnected thereto) positioned in the registered position relative to the imaging field of the CT imaging device and the predetermined frame of reference (e.g. maintained in the registered position after initial positioning), to obtain a third set of 3D image data of the VOL The third set of 3D image data may include:
image data corresponding with the at least one anatomical structure located in the VOI and different than the TMOI;
image data corresponding with the plurality of fiducial markers; and,
image data corresponding with one of tissue removal and tissue treatment of at least a portion of the TMOI utilizing the at least one or more needle(s).

The third processing may include third reconstructing the third set of 2D image data sets to obtain the third set of 3D image data (e.g. reconstructing by a computer processor configured by executable computer code comprising an image reconstruction algorithm stored in non-transient memory).The method may further include third generating third image display data utilizing the third set of 3D image data (e.g. generating by a computer processor configured by executable computer code stored in non-transient memory), wherein the third image display data may be provided for image display at a user interface in response to receipt of input indicative of at least one user-selected view of the VOI at the user interface. The third generating may include the provision of third image display data in response to user input indicative of one or multiple user-selected views of the VOI at a user interface. The user selected view(s) may be two-dimensional and/or three-dimensional. The displayed view(s) may comprise a plurality of selected cross-sectional 2D views across the VOI and/or a plurality of selected perspective 3D views of the VOI, including dynamically generated 3D panning views. For such purposes, method may further include configuring the user interface to provide for user input and selection of one or multiple views of the VOI for display in relation to the third set of 3D image data (e.g. configuring by configuration of a computer processor by executable code).

In some implementations, the third processing may further include second segmenting the reconstructed third set of 2D image data sets to obtain the third set of 3D image data (e.g. segmenting by a computer processor configured by executable computer code stored in non-transient memory). Such segmentation may be provided to provide enhanced image differentiation at different volume borders within the VOI at which tissue characteristics differ but may not be otherwise readily visible as such, and/or across tissue volumes within the VOI and within which tissue characteristics are similar but may not be otherwise readily visible as such, thereby enhancing the ability to visually identify a TMOI and precise features thereof, including for example, volume border features and intra-volume vascular features (e.g. a periphery of an ablated tissue volume or of a volume from which tissue has been removed for analysis).

In some approaches, the third reconstructing and second segmenting of the third processing may be completed together. In other approaches, the third reconstructing may be completed, followed by the second segmenting.

In the later regard, the third reconstructing may be completed and the reconstructed third set of 2D image data sets may be provided for use as the third set of 3D image data in the third generating step, wherein corresponding third image display data may be provided for image display at the user interface. In turn, the second segmenting may include second applying a predetermined segmentation algorithm (e.g. the same predetermined segmentation algorithm as applied in the first applying of the first processing step) to the reconstructed third set of 2D image data sets (e.g. applying by a computer processor configured by executable computer code comprising the predetermined segmentation algorithm stored in non-transient memory), in response to input of at least one user-selected location relative to at least one user-selected view of the VOI displayed at the user interface (e.g. selected by a user at the user interface). Such user-selected location, or “seed” location, may be a user selected volume within the VOI that a user has identified in the displayed view as corresponding with the TMOI (e.g. a volume including or otherwise corresponding with at least a portion of the TMOI). In turn, the predetermined segmentation algorithm may provide for enhanced image differentiation of a volume determined by the segmentation algorithm as having characteristics corresponding with those of the volume (e.g. tissue volume) located at the user-selected “seed” location. That is, the volume and/or volume borders of the determined volume may be enhanced, or otherwise differentiated, in the segmented third set of 3D image data and provided for use in the third generating step, wherein upon display of one or multiple 2D and/or 3D views at the user interface, a user may visually assess the determined volume that is enhanced in the displayed view(s), e.g. including dynamically generated 3D panning views, whereupon a user may select the segmented third set of 3D image data for further use.

The method may further include second registering of the third set of 3D image data of the VOI with one of the first and second sets of 3D image data sets of the VOI to obtain a second registered 3D image data set of the VOL For example, the second registering may register the segmented first set of 3D image data with the segmented third set of 3D image data. In some implementations, the second registering may include deformable 3D registration of the third set of 3D image data of the VOI with the one of the first and second sets of 3D image data sets of the VOI utilizing at least the corresponding image data corresponding with the at least one anatomical structure and optionally the image data corresponding with the plurality of fiducial markers, to obtain the second registered 3D segmentation image data set of the VOI.

After the second registering, the method may include fourth generating fourth image display data utilizing the second registered 3D image data set. The fourth image display data may be provided for display in response to input indicative of at least one or more user-selected view(s) of the VOI at the user interface. The user selected view(s) may be two-dimensional and/or three-dimensional. The displayed view(s) may comprise a plurality of selected cross-sectional 2D views across the VOI and/or a plurality of selected perspective 3D views of the VOI, including dynamically generated 3D panning views, thereby allowing a user to visually confirm one of the removal of one more desired tissue sample(s) from the TMOI or the desired treatment of the TMOI (e.g. ablative treatment of cancerous or otherwise diseased tissue), including desired margins about the TMOI. For such purposes, method may further include configuring the user interface to provide for user input and selection of one or multiple views of the VOI for display in relation to the second registered 3D image data (e.g. configuring by configuration of a computer processor by executable code). In the event a user determines that the desired tissue sample(s) was not removed or that the desired tissue treatment was not achieved, the user interface may be configured to receive user input to repeat the determining step and any of the steps described herein that follow the determining step. In contemplated system embodiments, the method elements described herein may be provided by one or more computer processor(s) configured by executable computer code comprising one or more software module(s) stored in non-transitory memory, and one or more user interface(s) configurable by the computer processor(s) to display image data and receive user input as described herein. Such system elements may be operatively interconnected with a CT imaging device to receive and process 2D image data sets for use in the described method elements, and with a robotically-positionable needle guide via a controller to provide for enhanced positioning of the robotically-positionable needle guide in an automated manner, as described in the method elements presented herein.

Numerous additional features and advantages of the present invention will become apparent to those skilled in the art upon consideration of the embodiment descriptions provided hereinbelow.

### BRIEF DESCRIPTION OF THE DRAWINGS

FIG. 1 is a schematic view of one embodiment of a system for use in positioning a needle guide relative to a volume-of-interest.

FIGS. 2A and 2B illustrate one embodiment of a method for use in positioning a needle guide relative to a volume-of-interest.

### DETAILED DESCRIPTION

The following description is not intended to limit the invention to the forms disclosed herein. Consequently, variations and modifications commensurate with the following teachings, skill and knowledge of the relevant art, are within the scope of the present invention. The embodiments described herein are further intended to explain modes known of practicing the invention and to enable others skilled in the art to utilize the invention in such, or other embodiments and with various modifications required by the particular application(s) or use(s) of the present invention.

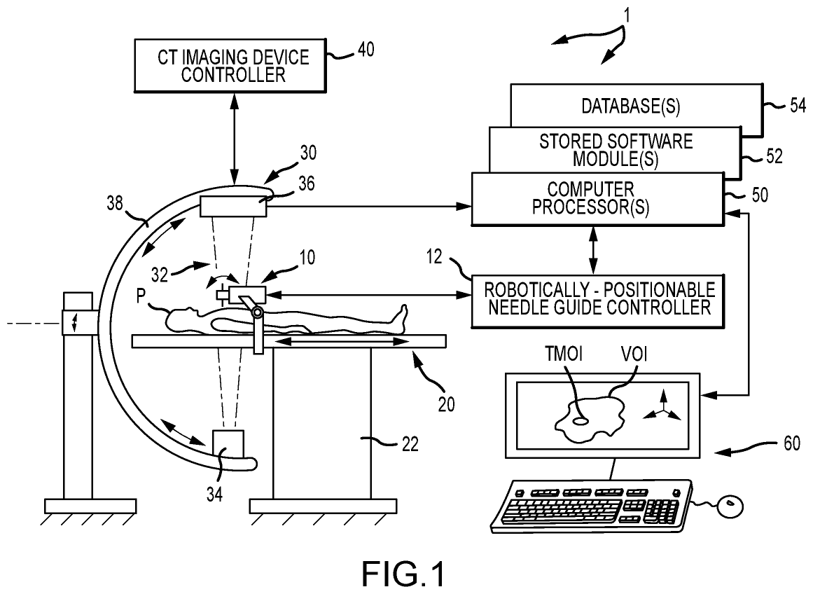

FIG. 1 illustrates one embodiment of a system 1 for use in positioning a needle guide relative to a tissue volume-of-interest (VOI) within a given patient P. In the illustrated embodiment, the needle guide is provided as part of a robotically-positionable needle guide 10 that may be supportably interconnected to/disconnected from a patient support platform 20 at a selectable location, e.g. a selected one of a continuum of locations along a horizontal edge extending along a length of the patient support platform 20. In turn, the patient support platform 20 (i.e. with a patient P positioned thereupon and the robotically-positionable needle guide 10 interconnected thereto) may be positioned relative to a computed tomography (CT) imaging device 30 at a registered position relative to a CT imaging field 32 of the CT imaging device 30 and a corresponding predetermined field of reference.

The CT imaging device 30 may comprise an x-ray source 34 and x-ray detector 36 supportably interconnected in opposing relation to a C-arm 38 of the CT imaging device 30, so as to define the CT imaging field 32 therebetween. The x-ray source 34 and x-ray detector 36 may be provided to rotate the CT imaging field 32 about the tissue volume-of-interest (VOI) of the patient P supported by the patient support platform 20. In that regard, the patient support platform 20 may be moveably supported on a stationary pedestal 22 in a manner that allows the patient support platform 20 to be selectively retracted away from the CT imaging field 32 to facilitate patient positioning on the patient support platform 20 and interconnection of the robotically-positionable needle guide thereto, then selectively advanced to the registered position in the CT imaging field 32. In some arrangements, the CT imaging device 30 may be advantageously provided for cone beam CT.

The CT imaging device 30 may be controlled by a CT imaging device controller 40. In that regard, a user may utilize an interface of the CT imaging device controller 40 to establish the desired parameters for one or more CT imaging instance(s) of a given patient tissue VOI.

The system 1 may include at least one or more computer processor(s) 50, configurable by executable computer code, including executable computer code comprising one or more software module(s) 52 stored in non-transitory memory, and one or more user interface(s) 60 interconnected Oto the computer processor(s). The computer processor(s) 50 may be interconnected to the CT imaging device 30 to receive two-dimensional (2D) image data set(s) of the patient tissue VOI obtained by the CT imaging device 30 in corresponding relation to separate instances of CT imaging (e.g. CT imaging without patient injection of a contrast bolus, CT imaging with patient injection of a contrast bolus, CT imaging at different power settings (e.g. different kV levels), and/or combinations thereof). In turn, the executable computer code may be provided to configure the computer processor(s) 50 to process the 2D image data sets and generate or otherwise determine additional related data, including image display data and needle guide positioning data, as described herein. The 2D image data sets and related data may be stored in one or more database(s) 54 by the computer processor(s) 50.

The computer processor(s) 50 may also be interconnected to a robotically-positionable needle guide controller 12 that controls the automated positioning and operation of robotically-positionable needle guide 10. In turn, needle guide positioning data may be provided by the computer processor(s) 50 to the robotically-positionable needle guide controller 12 for use in automated positioning of robotically-positionable needle guide 10, as described herein. The controller 12 may include one or more computer processor(s), associated memory, and a user interface for establishing automated operation parameters. A plurality of radio-opaque fiducial markers may be supportably interconnected to the robotically-positionable needle guide 10 in predeterminable co-relation at different corresponding locations in radiolucent portions thereof. In turn, the fiducial markers may be identified in the 2D image data sets, wherein the locations thereof may be determined and utilized in an automated manner in conjunction with the automated positioning of the robotically-positionable needle guide 10. In one arrangement, the robotically-positionable needle guide 10 and controller 12 may comprise the “iSYS 1 Navi+ System” product offered by iSYS Medizintechnik GmbH, Bergwerksweg 21, 6370 Kitzbuhel/Austria.

As indicated, the executable computer code may be provided to configure the computer processor(s) 50 to process the 2D image data sets. More particularly, in conjunction with contemplated tissue biopsy and tissue ablation procedures, the executable computer code may be provided to configure the computer processor(s) 50 to process one or more first set(s) of 2D image data sets to obtain one or more first set(s) of 3D image data of the VOL Such processing may entail reconstruction of the one or more first set(s) of 2D image data sets, utilizing a reconstruction algorithm of stored software module(s) 52, to obtain the one or more first set(s) of 3D image data. Further, the executable computer code may be provided to configure the computer processor(s) 50 to utilize the one or more first set(s) of 3D image data to generate first image display data of the VOI for image display at the user interface(s) 60, and to configure the user interface(s) 60 to receive input of user-selected 2D and/or 3D view(s) of the VOI for image display, including dynamically generated 3D panning views, as described herein.

The displayed view(s) allow a user to identify a tissue mass of interest (TMOI) within the VOI of the patient P. The TMOI may comprise tissue to be biopsied for analysis (e.g. cytological tissue analysis to determine if the TMOI is diseased), or the TMOI may comprise diseased tissue to be ablated.

To enhance the ability of a user to identify the TMOI, the executable computer code may be provided to configure the computer processor(s) 50 to segment one or more reconstructed first set(s) of 3D image data, utilizing a segmentation algorithm of stored software module(s) 52, in response to input of at least one user-selected location relative to at least one user-selected view of the VOI displayed at the user interface 60 (e.g. selected by a user at the user interface 60). Such user-selected location, or “seed” location, may be a user-selected volume within the VOI that a user has identified in the displayed view as corresponding with the TMOI (e.g. a volume including at least a portion of the TMOI). In turn, and as described herein, the predetermined segmentation algorithm may provide for enhanced image differentiation of a tissue volume determined by the segmentation algorithm as having characteristics corresponding with those of the tissue located at the user-selected “seed” location.

The executable code may be further provided to configure the user interface 60 to provide for user selection of a first set of 3D image data (e.g. a segmented first set of 3D image data) for use in determining needle guide positioning data. In turn, the executable computer code may be further provided to configure the computer processor(s) 50 to determine needle guide positioning data indicative of one or more desired needle placement location(s) in the VOL For such purposes, the executable computer code may be further provided to configure the computer processor(s) 50 to configure the user interface(s) 60 to receive input of user-selected needle placement location(s) in relation to a user-selected view of the VOI displayed at the user interface 60. In that regard, a user may select one or more needle placement location(s) at user interface 60 that the user has determined desirable for guiding a tissue removal device or tissue treatment device to a desired location. The needle guide positioning data may include:
data indicative of user-selected needle insertion and inserted needle tip locations (e.g. 3D coordinates of each location relative to the predetermined frame of reference) corresponding with each of the one or more user-selected needle placement location(s) (i.e. for each a given needle, the 3D coordinates if the locations at which a user selects for needle entry in to patient tissue and an “end-point” needle tip location within the patient tissue); and,
data indicative of locations of the plurality of fiducial markers (e.g. 3D coordinates of each location relative to the predetermined frame of reference) in corresponding relation to each of the one or more user-selected needle placement location(s).

In turn, and as noted, the needle guide positioning data may be provided to the robotically positionable needle guide controller 12 for automated positioning of the robotically-positionable needle guide 10 relative to the VOI, free from manual positioning thereof. As such, the automated positioning may be completed with the patient support platform 20 (i.e. with the patient positioned thereupon and the robotically-positionable needle guide interconnected thereto) located at the registered position relative to the CT imaging field 32 of the CT imaging device 30 and the predetermined frame of reference (e.g. maintained in the registered position after initial positioning). The automated positioning of the robotically-positionable needle guide 10 may be provided to locate the robotically-positionable needle guide 10 so that the one or more needle(s) may be guided thereby to the corresponding one or more user-selected needle placement location(s) (e.g. corresponding with the 3D coordinates of the user-selected needle insertion and inserted needle tip locations). Needle placement utilizing the robotically-positionable needle guide 10 may be automated, manual, and/or a combination thereof. In some applications (e.g. tissue biopsy and tissue treatment applications), the automated positioning of the robotically-positionable needle guide may be provided to successively locate the robotically-positionable needle guide to a plurality of different locations so that each of a plurality of needles may be successively guided thereby to a corresponding plurality of different user-selected needle placement locations.

In conjunction with contemplated procedures, the executable computer code may be provided to configure the computer processor(s) 50 to process a second set of 2D image data obtained after needle placement so as to obtain a second set of 3D image data of the VOL Such processing may entail reconstruction of the second set of 2D image data sets, utilizing the reconstruction algorithm of stored software module(s) 52, to obtain the second set of 3D image data. Further, the executable computer code may be provided to configure the computer processor(s) 50 to register the first and second sets of 3D image data to obtain a first registered 3D image data set of the VOL Such registration may include deformable 3D registration processing of the first and second sets of 3D image data, as described herein.

In turn, the executable computer code may be provided to configure the computer processor(s) 50 to utilize the first registered 3D image data set to generate second image display data of the VOI for image display at the user interface(s) 60, and to configure the user interface(s) 60 to receive input of user-selected 2D and/or 3D view(s) of the VOI for image display, including dynamically generated 3D panning views, as described herein. The second image display data may be displayed so that a user may visually determine the desirability of the location of the needle(s) in the VOL In turn, if the user is dissatisfied with the location of the one needle(s), further needle guide positioning data may be determined and utilized for additional needle placement, as described herein.

In conjunction with contemplated procedures, after acceptable positioning of the needle(s), a user may proceed with the tissue biopsy or tissue ablation procedure, wherein a tissue removal device or tissue ablation device may be guided by the needle(s) to one or more locations for completion of the procedure. In turn, the executable computer code may be provided to configure the computer processor(s) 50 to process a third set of 2D image data obtained after the given procedure so as to obtain a third set of 3D image data of the VOL Such processing may entail reconstruction of the third set of 2D image data sets, utilizing the reconstruction algorithm of stored software module(s) 52, to obtain the third set of 3D image data.

In turn, the executable computer code may be provided to configure the computer processor(s) 50 to utilize the third 3D image data set to generate third image display data of the VOI for image display at the user interface(s) 60, and to configure the user interface(s) 60 to receive input of user-selected 2D and/or 3D view(s) of the VOI for image display, including dynamically generated 3D panning views, as described herein. The third image display data may be displayed so that a user may visually determine the acceptability of the removed tissue sample(s) or of the treated tissue (e.g. including the ability to visually confirm complete treatment of a diseased tissue volume with desired margins).

To enhance the ability of a user to determine the acceptability of the removed tissue sample(s) or of the treated tissue, the executable computer code may be provided to configure the computer processor(s) 50 to segment the third set of 3D image data, utilizing the segmentation algorithm of stored software module(s) 52, in response to input of at least one user-selected location relative to at least one user-selected view of the VOI displayed at the user interface 60 (e.g. selected by a user at the user interface 60). Such user-selected location, or “seed” location, may be a user-selected volume within the VOI that a user has identified in the displayed view as corresponding with the desired treatment of tissue. In turn, and as described herein, the predetermined segmentation algorithm may provide for enhanced image differentiation of a tissue volume determined by the segmentation algorithm as having characteristics corresponding with those of the treated tissue located at the user-selected “seed” location.

In turn, the executable computer code may be provided to configure the computer processor(s) 50 to register the segmented first and third sets of 3D image data to obtain a second registered 3D image data set of the VOL Such registration may include deformable 3D registration processing of the first and third sets of 3D image data, as described herein.

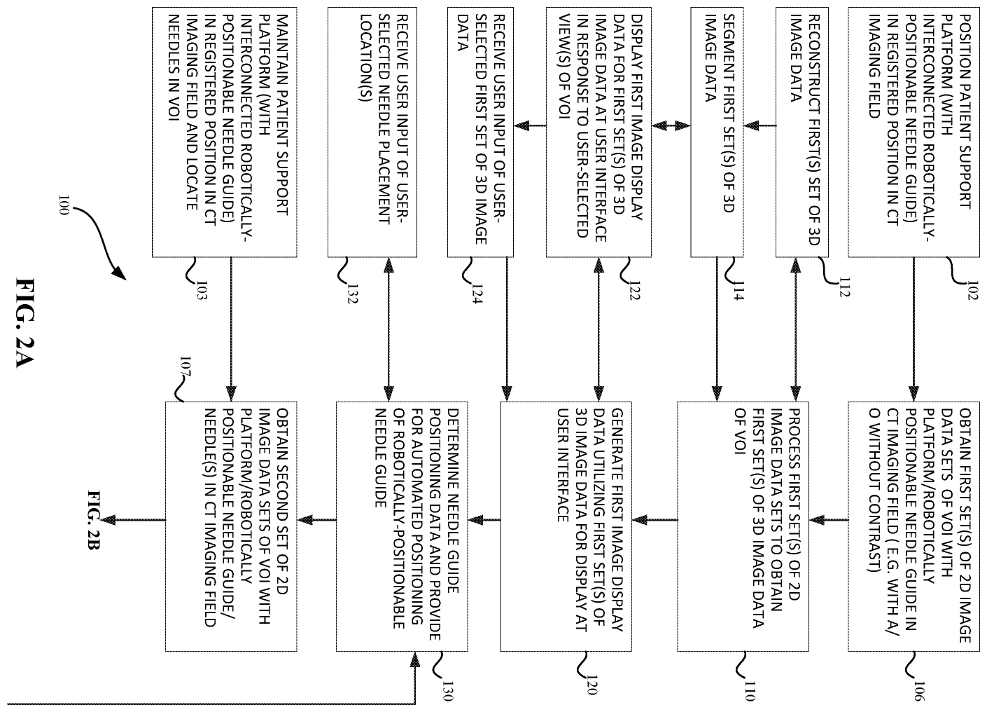

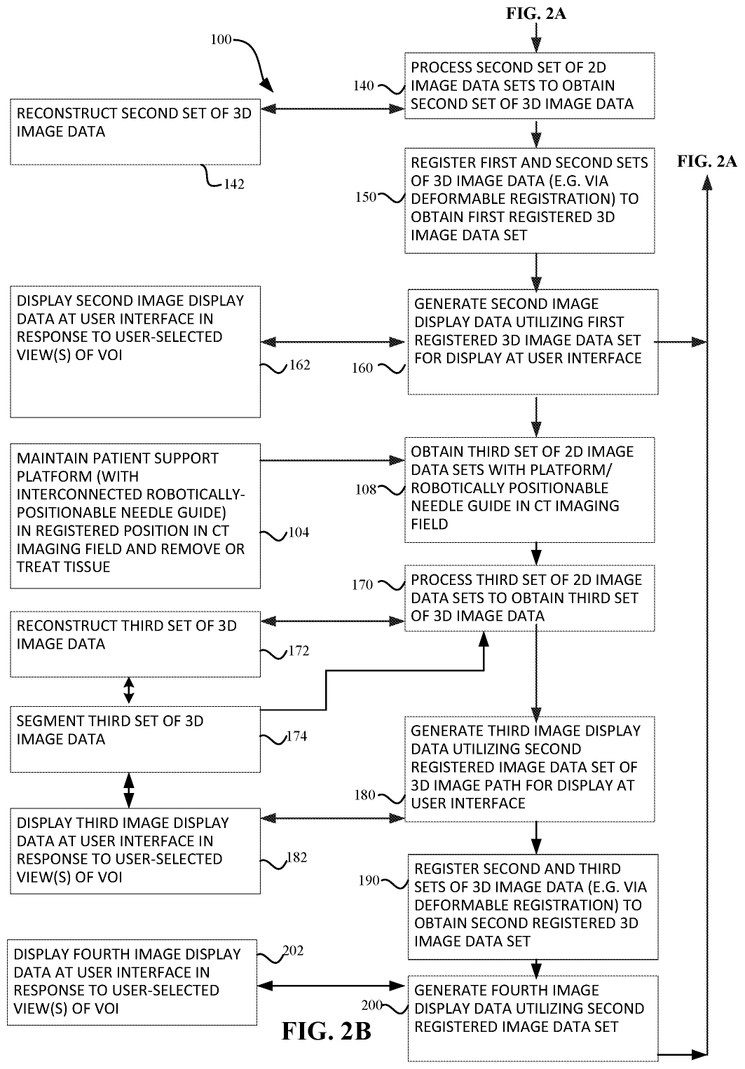

In turn, the executable computer code may be provided to configure the computer processor(s) 50 to utilize the second registered 3D image data set to generate fourth image display data of the VOI for image display at the user interface(s) 60, and to configure the user interface(s) 60 to receive input of user-selected 2D and/or 3D view(s) of the VOI for image display, including dynamically generated 3D panning views, as described herein. The fourth image display data may be displayed so that a user may visually confirm the acceptability of the removed tissue sample(s) or of the treated tissue (e.g. visual confirmation of complete treatment of a diseased tissue volume with desired margins). In turn, if the user is dissatisfied, further needle guide positioning data may be determined and utilized for additional needle placement, followed by additional tissue removal or tissue treatment, as described herein. Reference is now made to FIGS. 2A and 2B which illustrate an embodiment of a method (100) for use in positioning a needle guide relative to a tissue volume-of-interest (VOI). The method (100) may be implemented in various system embodiments, including the embodiment of system 1 described above.

The method (100) may include first processing (110) one or more first set(s) of two-dimensional (2D) image data to obtain at least one or more first set(s) of three-dimensional (3D) image data of the VOI (e.g. processing by a computer processor 50 configured by executable computer code stored in non-transient memory). The one or more first set(s) of 2D image data sets may be obtained (104) by a computed tomography (CT) imaging device (e.g. CT imaging device 30), after positioning (102) of a patient support platform (e.g. patient support platform 20), with a robotically-positionable needle guide (e.g. robotically-positionable needle guide 10) interconnected thereto and a patient positioned thereupon, in a registered position relative to an imaging field of the CT imaging device and a corresponding predetermined frame of reference. In some arrangements, a plurality of first sets of 2D image data sets may be obtained (104), each after positioning (102), and processed (110) to obtain a corresponding plurality of first sets of 3D image data in corresponding relation to a plurality of different imaging instances, e.g. CT imaging of the VOI without an injection of a contrast bolus, CT imaging of the VOI with injection of a contrast bolus, CT imaging of the VOI at different power settings (e.g. different kV levels), and/or combinations thereof. The first processing (110) may include first reconstructing (112) the one or more first set(s) of 2D image data sets to obtain reconstructed one or more first set(s) of 2D image data sets as the one or more first set(s) of 3D image data (e.g. reconstructing by a computer processor configured by executable computer code stored in non-transient memory).

Prior to positioning (102), the patient may be located on and the robotically-positionable needle guide may be interconnected to the patient support platform (e.g. supportably interconnected for movement with the patient support platform) at an initial location of the patient support platform that is at least partially or entirely outside of the imaging field of the CT imaging device, wherein the support platform is then subsequently positioned (102) at the registered position relative to the imaging field of the CT imaging device and the predetermined frame of reference. In the later regard, the robotically-positionable needle guide may be advantageously operable for automated positioning relative to the VOI while the support platform is located at the registered position relative to and partially within the CT imaging field, free from manual positioning thereof. In some arrangements, the robotically-positionable needle guide may be provided so that portions thereof that may be locatable within the CT imaging field may be radiolucent, thereby facilitating in-field positioning of the robotically-positionable needle guide during imaging procedures.

The one or more first set(s) of 3D image data obtained in the first processing (110) may each include:
image data corresponding with a tissue mass-of-interest (TMOI) located in the VOI;
image data corresponding with at least one anatomical structure located in the VOI and different than the TMOI; and
image data corresponding with a plurality of fiducial markers, wherein the robotically-positionable needle guide and the plurality of fiducial markers are disposed in predeterminable relation.
   In some arrangements, the plurality of fiducial markers may be supportably interconnected to the robotically-positionable needle guide in different corresponding locations (e.g. as part of an interconnected assembly and in radiolucent portions thereof).

The method (100) may further include first generating (120) first image display data utilizing the one or more first set(s) of 3D image data (e.g. generating by a computer processor 50 configured by executable computer code stored in non-transient memory), wherein the first image display data may be provided for image display (122) in response to receipt of input indicative of at least one user-selected view of the VOI at a user interface. In that regard, for each first set of 3D image data, the first generating (120) may correspondingly include the provision of first image display data for display (122) in response to input indicative of multiple user-selected views of the VOI at a user interface. The user selected view(s) may be two-dimensional and/or three-dimensional. The displayed view(s) may comprise a plurality of selected cross-sectional 2D views across the VOI and/or a plurality of selected perspective 3D views of the VOI, including dynamically generated 3D panning views. For such purposes, the method may further include configuring the user interface to provide for user input and selection of one or multiple views of the VOI for display in relation to each first set of 3D image data (e.g. configuring by configuration of a computer processor 50 by executable code). Where a plurality of first sets of 3D image data are generated/viewed, the method may further include receiving (124) input indicative of a user-selected set for further use as the first set of 3D image data. In turn, the method may further include configuring the user interface to provide for the user input and selection of a user-selected set for further use as the first set of 3D image data (e.g. configuring by configuration of a computer processor 50 by executable code).

In contemplated implementations, the first processing (110) may further include first segmenting (114) one or more of the reconstructed one or more first sets(s) of 2D image data sets to obtain the one or more first set(s) of 3D image data (e.g. segmenting by a computer processor 50 configured by executable computer code stored in non-transient memory). Such segmentation may be provided to provide enhanced image differentiation at different volume borders within the VOI at which tissue characteristics differ but may not be otherwise readily visible as such, and/or across tissue volumes within the VOI and within which tissue characteristics may be similar but not otherwise readily visible as such, thereby enhancing the ability to visually identify a TMOI and precise features thereof, including for example, volume border features and intra-volume vascular features(e.g. a periphery of a tissue volume corresponding with a cancerous tumor or other diseased tissue).

In some approaches, the first reconstructing (112) and first segmenting (114) may be completed together. In other approaches, the first reconstructing (112) may be completed, followed by the first segmenting (114).

In the later regard, the first reconstructing (112) may be completed and the reconstructed one or more first set(s) of 2D image data sets may be provided for use as the one or more first set(s) of 3D image data in the first generating (120), wherein corresponding first image display data may be provided for image display (122) at the user interface. In turn, the first segmenting (114) may include the application of a predetermined segmentation algorithm to one or more of the reconstructed one or more first set(s) of 2D image data (e.g. applying by a computer processor 50 configured by executable computer code comprising the predetermined segmentation algorithm stored in non-transient memory), in response to input of at least one user-selected location relative to at least one user-selected view of the VOI displayed at the user interface (e.g. selected by a user at the user interface). Such user-selected location, or “seed” location may be a user-selected volume within the VOI that a user has identified in the displayed view as corresponding with the TMOI (e.g. a volume including all or at least a portion of the TMOI). In turn, the predetermined segmentation algorithm may provide enhanced image differentiation a tissue volume having characteristics corresponding with those of the tissue located at the user-selected location. For example, the volume and/or volume borders of the determined tissue volume may be enhanced, or otherwise differentiated, in the segmented corresponding first set of 3D image data and provided for use in the first generating step, wherein upon display of one or multiple 2D and/or 3D views at the user interface, a user may visually assess the determined tissue volume that is enhanced in the displayed view(s), e.g. including dynamically generated 3D panning views, whereupon a user may select the segmented corresponding first set of 3D image data as the user-selected first set of 3D image date for further use.

In conjunction with such first segmenting (114), the method may further include receiving input indicative of a user-selected location, or volume, for segmentation. In turn, the method may further include configuring the user interface to provide for the user input and selection of the one or more user-selected location(s) for use in the application of the predetermined segmentation algorithm (e.g. configuring by configuration of a computer processor by executable code). As noted, pursuant to the first segmenting (114), the segmented one or more first set(s) of 3D image data may be utilized in the first generating (120) to provide corresponding first image display data to the user interface, wherein one of the segmented one or more first set(s) of 3D image data may be selected for use or otherwise used in the method. In that regard, the method may include configuring the user interface to provide for user selection of a segmented first set of 3D image data for further use (e.g. configuring by configuration of a computer processor by executable code).

The method may further include first determining (130) needle guide positioning data utilizing the one or user-selected first set of 3D image data. In that regard, the first determining (130) may be completed upon receiving (132) input indicative of one or more user-selected needle placement location(s) relative to at least one or more corresponding user-selected view(s) across the VOI displayed at the user interface (e.g. determining by a computer processor 50 configured by executable computer code stored in non-transient memory) in conjunction with the displaying (122). In that regard, the method may include configuring the user interface so that a user may select one or more needle placement location(s) that the user has determined desirable for guiding a tissue removal device or tissue treatment device to a desired location (e.g. configuring by a computer processor 50 configured by executable computer code stored in non-transient memory). The needle guide positioning data may include:
data indicative of user-selected needle insertion and inserted needle tip locations (e.g. 3D coordinates of each location relative to the predetermined frame of reference) corresponding with each of the one or more user-selected needle placement location(s) (i.e. for each a given needle, the 3D coordinates of the locations at which a user selects for needle entry in to patient tissue and an “end-point” needle tip location within the patient tissue); and,
data indicative of locations of the plurality of fiducial markers (e.g. 3D coordinates of each location relative to the predetermined frame of reference) in corresponding relation to each of the one or more user-selected needle placement location(s).
   In turn, the needle guide positioning data may be provided for automated positioning of the robotically-positionable needle guide relative to the VOI, free from manual positioning thereof. As such, in response to the provision of the needle guide positioning data, the automated positioning may be advantageously completed with the patient support platform (i.e. with the patient positioned thereupon and the robotically-positionable needle guide interconnected thereto) located at the registered position relative to the imaging field of the CT imaging device and the predetermined frame of reference (e.g. maintained in the registered position after initial positioning), thereby facilitating the realization of enhanced positioning of the one or more needle(s) in relation to the user-selected needle placement location(s).

In contemplated embodiments, one or more needle(s) may then be located in the VOI in corresponding relation to the one or more user-selected needle placement location(s) comprising the needle guide positioning data. Such needle placement may be automated, manual, and/or a combination thereof. For example, the automated positioning of the robotically-positionable needle guide may be provided to locate the robotically-positionable needle guide so that the one or more needle(s) may be guided thereby to the corresponding one or more user-selected needle placement location(s) (e.g. corresponding with the 3D coordinates of the user-selected needle insertion and inserted needle tip locations). In some applications (e.g. tissue treatment applications), the automated positioning of the robotically-positionable needle guide may be provided to successively locate the robotically-positionable needle guide to a plurality of different locations so that each of a plurality of needles may be successively guided thereby to a corresponding plurality of different user-selected needle placement locations.

The method may further include, e.g. after positioning of the one or more needle(s) at the corresponding at least one or more user-selected needle placement location(s), second processing (140) at least one second set of 2D image data sets obtained via second obtaining (107) in a separate imaging instance by the computed tomography (CT) imaging device (e.g. processing by a computer processor 50 configured by executable computer code stored in non-transient memory), with the patient support platform (i.e. with the patient positioned thereupon and the robotically-positionable needle guide interconnected thereto) maintained (103) in the registered position within and relative to the imaging field of the CT imaging device and the predetermined frame of reference, to obtain a second set of 3D image data of the VOL The second set of 3D image data may include:
image data corresponding with the tissue mass-of-interest (TMOI) located in the VOI;
image data corresponding with the at least one anatomical structure located in the VOI and different than the TMOI;
image data corresponding with the plurality of fiducial markers; and,
image data corresponding with the one or more needle(s) located in to the VOI utilizing the robotically-positionable needle guide after the automated positioning of the robotically-positionable needle guide relative to the VOI.

The method may further include first registering (150) the first and second sets of 3D image data of the VOI to obtain a first registered 3D image data set of the VOI (e.g. registering by a computer processor 50 configured by executable computer code stored in non-transient memory). In some arrangements, the first registering (150) may include deformable 3D registration processing of the first and second sets of 3D image data of the VOI (e.g. processing by a computer processor configured by executable computer code stored in non-transient memory), utilizing at least the corresponding image data of the first and second sets of 3D image data corresponding with the at least one anatomical structure, and optionally the image data corresponding with the plurality of fiducial markers, to obtain the first registered 3D image data set of the VOL Such deformable registration may account for relative positional changes of common features of the first and second sets of 3D image data of the VOI.

After the first registering (150), the method may include second generating (160) second image display data utilizing the first registered 3D image data set (e.g. generating by a computer processor 50 configured by executable computer code stored in non-transient memory), wherein the second image display data is provided for image display (162) in response to input indicative of at least one user-selected view of the VOI at the user interface. The second generating (160) may include the provision of second image display data for image display (162) in response to input indicative of multiple different user-selected views of the VOI at a user interface. The user selected view(s) may be two-dimensional and/or three-dimensional. The displayed view(s) may comprise a plurality of selected cross-sectional 2D views across the VOI and/or a plurality of selected perspective 3D views of the VOI, including dynamically generated 3D panning views. For such purposes, the method (100) may further include configuring the user interface to provide for user input and selection of one or multiple views of the VOI for display in relation to the first registered 3D image data (e.g. configuring by configuration of a computer processor by executable code).

The second image display data may be displayed so that a user may visually determine the desirability of the location of the one or more needle(s) in the VOL In turn, if the user is dissatisfied with the location of the one or more needle(s), the first determining (130) needle guide positioning data step may be repeated in response to the receipt of input at user interface that is indicative of one or more revised user-selected needle placement location(s) relative to at least one user-selected view across the VOI displayed at the user interface to obtain the needle guide positioning data. In turn, the second obtaining (107), second processing (140), first registering (150), and second generating (160) steps may be repeated.

As noted, automated positioning of the robotically-positionable needle guide may be advantageously completed while the support platform (i.e. with the patient positioned thereupon and the robotically-positionable needle guide interconnected thereto) is located at the registered position relative to the imaging field of the CT imaging device and the predetermined field of reference (e.g. maintained in the registered position after initial positioning (102)). In turn, the first processing (110), first generating (120), first determining (130), second processing (140), first registering (150) and second generating (160) steps may be completed with the patient support platform positioned at the registered position relative to the imaging field of the CT imaging device and the predetermined field of reference a single time.

In contemplated embodiments, after the second generating (160), one or more tissue removal or tissue treatment device(s) may be located by the one or more needle(s) located in the VOI for tissue removal from or tissue treatment of the TMOI. For example, the tissue removal or tissue treatment device(s) may be advanced in to an exposed, open end of the one or more needle(s) and guided thereby for tissue removal from or tissue treatment of the TMOI in an automated, manual, and/or semi-automated manner.

In contemplated embodiments, after the tissue removal from or tissue treatment of the TMOI, the method may further include third processing (170) at least a third set of 2D image data sets obtained via third obtaining (108) in a separate imaging instance by the computed tomography (CT) imaging device (e.g. processing by a computer processor configured by executable computer code stored in non-transient memory), with the patient support platform (i.e. with the patient positioned thereupon and the robotically-positionable needle guide interconnected thereto) maintained (104) in the registered position relative to the imaging field of the CT imaging device and the predetermined frame of reference (e.g. maintained in the registered position after initial positioning (102)), to obtain a third set of 3D image data of the VOL The third set of 3D image data may include:
image data corresponding with the at least one anatomical structure located in the VOI and different than the TMOI;
image data corresponding with the plurality of fiducial markers; and,
image data corresponding with one of tissue removal and tissue treatment of at least a portion of the TMOI utilizing the at least one or more needle(s).
   The third processing (170) may include third reconstructing (172) the third set of 2D image data sets to obtain the third set of 3D image data (e.g. reconstructing by a computer processor 50 configured by executable computer code comprising the image reconstruction code stored in non-transient memory).

The method may further include third generating (180) third image display data utilizing the third set of 3D image data (e.g. generating by a computer processor 50 configured by executable computer code stored in non-transient memory), wherein the third image display data may be provided for image display (182) at the user interface (e.g. user interface 60) in response to receipt of input indicative of at least one user-selected view of the VOI at the user interface. The third generating (180) may include the provision of third image display data in response to user input indicative of one or multiple user-selected views of the VOI at a user interface. The user selected view(s) may be two-dimensional and/or three-dimensional. The displayed view(s) may comprise a plurality of selected cross-sectional 2D views across the VOI and/or a plurality of selected perspective 3D views of the VOI, including dynamically generated 3D panning views. For such purposes, method may further include configuring the user interface to provide for user input and selection of one or multiple views of the VOI for display in relation to the third set of 3D image data (e.g. configuring by configuration of a computer processor by executable code).

In some implementations, the third processing (170) may further include second segmenting (174) the reconstructed third set of 2D image data sets to obtain the third set of 3D image data (e.g. segmenting by a computer processor configured by executable computer code stored in non-transient memory). Such segmentation may be provided to provide enhanced image differentiation at different volume borders within the VOI at which tissue characteristics differ but may not be otherwise readily visible as such, and/or across tissue volumes within the VOI and within which tissue characteristics are similar but may not be otherwise readily visible as such, thereby enhancing the ability to visually identify a TMOI and precise features thereof, including for example, volume border features and intra-volume vascular features (e.g. a periphery of an ablated tissue volume or of a volume from which tissue has been removed for analysis).

In some approaches, the third reconstructing (172) and second segmenting (174) of the third processing (170) may be completed together. In other approaches, the third reconstructing (172) may be completed, followed by the second segmenting (174).

In the later regard, the third reconstructing (172) may be completed and the reconstructed third set of 2D image data sets may be provided for use as the third set of 3D image data in the third generating step (180), wherein corresponding third image display data may be provided for image display (182) at the user interface. In turn, the second segmenting (174) may include second applying a predetermined segmentation algorithm (e.g. the same predetermined segmentation algorithm as applied in the first applying of the first processing step) to the reconstructed third set of 2D image data sets (e.g. applying by a computer processor configured by executable computer code comprising the predetermined segmentation algorithm stored in non-transient memory), in response to input of at least one user-selected location relative to at least one user-selected view of the VOI displayed at the user interface (e.g. selected by a user at the user interface). Such user-selected location, or “seed” location, may be a user selected volume within the VOI that a user has identified in the displayed view as corresponding with the TMOI (e.g. a volume including or otherwise corresponding with at least a portion of the TMOI). In turn, the predetermined segmentation algorithm may provide for enhanced image differentiation of a volume determined by the segmentation algorithm as having characteristics corresponding with those of the volume (e.g. tissue volume) located at the user-selected “seed” location. That is, the volume and/or volume borders of the determined volume may be enhanced, or otherwise differentiated, in the segmented third set of 3D image data and provided for use in the third generating step, wherein upon display of one or multiple 2D and/or 3D views at the user interface, a user may visually assess the determined volume that is enhanced in the displayed view(s), e.g. including dynamically generated 3D panning views, whereupon a user may select the segmented third set of 3D image data for further use.

The method may further include second registering (190) of the third set of 3D image data of the VOI with one of the first and second sets of 3D image data sets of the VOI to obtain a second registered 3D image data set of the VOI (e.g. registering by a computer processor 50 configured by executable computer code stored in non-transient memory. For example, the second registering (190) may register the segmented first set of 3D image data with the segmented third set of 3D image data. In some implementations, the second registering (190) may include deformable 3D registration of the third set of 3D image data of the VOI with the at least one of the first and second sets of 3D image data sets of the VOI utilizing at least the corresponding image data corresponding with the at least one anatomical structure to obtain the second registered 3D segmentation image data set of the VOI.

After the second registering (190), the method may include fourth generating (200) fourth image display data utilizing the second registered 3D image data set (e.g. generating by a computer processor 50 configured by executable computer code stored in non-transient memory). The fourth image display data may be provided for image display (202) in response to input indicative of at least one or more user-selected view(s) of the VOI at the user interface. The user selected view(s) may be two-dimensional and/or three-dimensional. The displayed view(s) may comprise a plurality of selected cross-sectional 2D views across the VOI and/or a plurality of selected perspective 3D views of the VOI, including dynamically generated 3D panning views, thereby allowing a user to visually confirm the desired obtainment of one or more tissue sample from the TMOI or to visually confirm the desired treatment of the TMOI (e.g. ablative treatment of cancerous or otherwise diseased tissue, including desired margins about the diseased tissue). For such purposes, method may further include configuring the user interface to provide for user input and selection of one or multiple views of the VOI for display in relation to the second registered 3D image data (e.g. configuring by configuration of a computer processor by executable code). In that regard, if a user is dissatisfied with the obtained tissue sample or treated TMOI, the user may choose to repeat one or more of the steps described hereinabove.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain known modes of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

What is claimed is:

1. A method for use in positioning a needle guide relative to a volume-of-interest (VOI), comprising:
first processing at least a first set of two-dimensional (2D) image data sets obtained by a computed tomography (CT) imaging device, with a patient support platform positioned in a registered position relative to an imaging field of the CT imaging device and a corresponding frame of reference, with a robotically positionable needle guide interconnected to and a patient supported by the patient support platform, to obtain a at least a first set of three-dimensional (3D) image data of the VOI, wherein the first set of 3D image data includes:
image data corresponding with a tissue mass-of-interest (TMOI) located in the VOI;
image data corresponding with at least one anatomical structure located in the VOI and different than the TMOI;
image data corresponding with a plurality of fiducial markers, wherein the robotically-positionable needle guide and the plurality of fiducial markers are disposed in predeterminable relation;
first generating first image display data utilizing the first set of 3D image data, wherein the first image display data is provided for image display in response to input indicative of at least one user-selected view of the VOI at a user interface;
first determining needle guide positioning data utilizing the first set of 3D image data in response to input indicative of at least one user-selected needle placement location relative to at least one user-selected view across the VOI displayed at the user interface, wherein the needle guide positioning data includes:
data indicative of user-selected needle insertion and inserted needle tip locations corresponding with said at least one user-selected needle placement location; and,
data indicative of locations of said plurality of fiducial markers;
wherein the needle guide positioning data is provided for automated positioning of the robotically-positionable needle guide relative to the VOI;
second processing at least a second set of 2D image data sets obtained by the computed tomography (CT) imaging device, with the patient support platform positioned in the registered position relative to the imaging field of the CT imaging device and the predetermined frame of reference, with the robotically-positionable needle guide interconnected to and the patient supported by the patient support platform, to obtain a second set of 3D image data of the VOI, wherein the second set of 3D image data includes:
image data corresponding with the tissue mass-of-interest (TMOI) located in the VOI;
image data corresponding with the at least one anatomical structure located in the VOI and different than the TMOI;
image data corresponding with the plurality of fiducial markers; and,
image data corresponding with at least one needle located in to the VOI after the automated positioning of the robotically-positionable needle guide relative to the VOI;
first registering the first and second sets of 3D image data of the VOI to obtain a first registered 3D image data set of the VOI; and,
second generating second image display data utilizing the first registered 3D image data set, wherein the second image display data is provided for image display in response to input indicative of at least one user-selected view of the VOI at the user interface.

2. The method of claim 1, wherein the first processing, first generating, first determining, second processing, first registering and second generating steps are completed with the patient support platform, with the robotically-positionable needle guide interconnected thereto and the patient supported thereby, positioned in the registered position relative to the imaging field of the CT imaging device and the predetermined frame of reference a single time.

3. The method of claim 1, wherein the first registering includes deformable 3D registration of the first and second sets of 3D image data of the VOI utilizing at least the corresponding image data corresponding with the at least one anatomical structure to obtain the first registered 3D image data set of the VOI.

4. The method of claim 1, wherein the first processing comprises:
first reconstructing the first set of 2D image data sets; and,
first segmenting the reconstructed first set of 2D image data sets to obtain the first set of 3D image data.

5. The method of claim 4, wherein the reconstructed first set of 2D image data sets is provided as the first set of 3D image data for use in the first generating first image display data, and wherein the first segmenting comprises:
first applying a predetermined segmentation algorithm to the reconstructed first set of 2D image data in response to input of at least one user-selected location relative to at least one user-selected view of the VOI displayed at the user interface.

6. The method of claim 4, wherein the first segmenting provides for enhanced visual differentiation of borders between different tissue types in the first image display data.

7. The method of claim 1, further comprising:
third processing at least a third set of 2D image data sets obtained by the computed tomography (CT) imaging device, with the patient support platform positioned in the registered position relative to the imaging field of the CT imaging device and the predetermined frame of reference, with the robotically-positionable needle guide interconnected to and the patient supported by the patient support platform, to obtain a third set of 3D image data of the VOI, wherein the third set of 3D image data includes:
image data corresponding with the tissue mass-of-interest (TMOI) located in the VOI;
image data corresponding with the at least one anatomical structure located in the VOI and different than the TMOI;
image data corresponding with the plurality of fiducial markers; and,
image data corresponding with one of removal and treatment of at least a portion of the TMOI utilizing the at least one needle; and,
third generating third image display data utilizing the third set 3D image data set, wherein the third image display data is provided for display in response to input indicative of at least one user-selected view at the user interface.

8. The method of claim 7, wherein the first processing, first generating, first determining, second processing, first registering, second generating, third processing and third generating are completed with the patient support platform, with the robotically-positionable needle guide interconnected thereto and the patient supported thereby, positioned in the registered position relative to the imaging field of the CT imaging device and the predetermined frame of reference a single time.

9. The method of claim 7, further comprising:
second registering the third set of 3D image data of the VOI with the first set of 3D image data to obtain a second registered 3D image data set of the VOI.

10. The method of claim 9, wherein the second registering includes deformable 3D registration of the third set of 3D image data of the VOI with the first set of 3D image data of the VOI utilizing at least the corresponding image data corresponding with the at least one anatomical structure to obtain the second registered 3D image data set of the VOI.

11. The method of claim 9, wherein the first processing comprises:
first reconstructing the first set of 2D image data sets; and,
first segmenting the reconstructed first set of 2D image data sets to obtain the first set of 3D image data; and,
wherein the third processing comprises:
third reconstructing the third set of 2D image data sets; and,
second segmenting the reconstructed third set of 2D image data sets to obtain the third set of 3D image data.

12. The method of claim 11, wherein the reconstructed first set of 2D image data sets is provided as the first set of 3D image data for use in the first generating first image display data, and wherein the first segmenting comprises:
first applying a predetermined segmentation algorithm to the reconstructed first set of 2D image data in response to input of at least one user-selected location relative to at least one user-selected view of the VOI displayed at the user interface; and, wherein the reconstructed third set of 2D image data sets is provided as the third set of 3D image data for use in the third generating third image display data, and wherein the second segmenting comprises:
second applying the predetermined segmentation algorithm to the reconstructed third set of 2D image data in response to input of at least one user-selected location relative to at least one user-selected view of the VOI displayed at the user interface.

13. The method of claim 12, wherein the first segmenting provides for enhanced visual differentiation of borders between different tissue types in the first image display data, wherein the second segmenting provides for enhanced visual differentiation of borders between different tissue types in the third image display data.

14. The method recited in claim 11, further comprising:
fourth generating fourth image display data utilizing the second registered 3D image data set, wherein the second image display data is provided for image display in response to input indicative of at least one user-selected view of the VOI at the user interface.

15. The method of claim 9, further comprising:
fourth generating fourth image display data utilizing the second registered 3D image data set, wherein the second image display data is provided for image display in response to input indicative of at least one user-selected view of the VOI at the user interface.

16. The method of claim 15, wherein the first processing, first generating, first determining, second processing, first registering, second generating, third processing, third generating, second registering and fourth generating are completed with the support platform, with the robotically-positionable needle guide interconnected thereto and the patient supported thereby, positioned in the registered position relative to the imaging field of the CT imaging device and the predetermined frame of reference a single time.

17. The method of claim 1, wherein after the first determining and before the second processing, the method further comprises:
automated positioning of the robotically-positionable needle guide relative to the VOI utilizing the needle guide positioning data, wherein the robotically-positionable needle guide is located to guide placement of at least one needle within the VOI.

18. The method of claim 17, wherein the first processing, first generating, first determining, automated positioning, second processing, first registering and second generating steps are completed with the patient support platform, with the robotically-positionable needle guide interconnected thereto and the patient supported thereby, positioned in the registered position relative to the imaging field of the CT imaging device and the predetermined frame of reference a single time.

19. A system for use in the method of claim 1, comprising:
one or more computer processor configurable by computer software stored in non-transitory memory to perform said first processing, first generating, first determining, second processing, first registering and second generating; and,
one or more user interface operatively interconnected to the one or more computer processor for image display using said first image display data and said second image display data, and for receipt of said input indicative of said at least one user-selected needle placement location.

20. The system of claim 19, wherein said one or more computer processor is operatively interconnected to the robotically-positionable needle guide to provide said needle guide positioning data for use in automated positioning of the robotically-positionable needle guide.
